(19) United States
(12) Patent Application Publication
(10) Pub. No.: US 2011/0287991 A1
(43) Pub. Date: Nov. 24, 2011

(54) METHOD FOR MANUFACTURING A BIOMASS-DERIVED METHYL METHACRYLATE

(75) Inventor: Jean-Luc Dubois, Millery (FR)

(73) Assignee: ARKEMA FRANCE, Colombes (FR)

(21) Appl. No.: 13/129,714

(22) PCT Filed: Nov. 17, 2009
(86) PCT No.: PCT/FR09/52198

§ 371 Date: Aug. 11, 2011

(30) Foreign Application Priority Data
Nov. 18, 2008 (FR) 0857812

(51) Int. Cl. C07C 67/343; C10M 129/70; C08L 27/06; C08L 33/12; C08F 20/14; C08F 220/14

(52) U.S. Cl. 508/463; 560/205; 526/329.7; 526/328.5; 525/309; 524/560

(57) ABSTRACT

Process for the manufacture of methyl methacrylate by reaction of methyl propionate with one from formaldehyde, a formaldehyde/methanol mixture and methylal, characterized in that at least a fraction of at least one reactant involved in this reaction was obtained by a reaction or a sequence of reactions starting from biomass.

The present invention relates to a process for the manufacture of a biomass-derived methyl methacrylate.

Methyl methacrylate is the starting material of numerous polymerization or copolymerization reactions.

It is the monomer for the manufacture of poly(methyl methacrylate) (PMMA), known under the Altuglas® and Plexiglas® trade names. It is provided in the form of powders, granules or sheets, the powders or granules being used for the molding of various items, such as items for the motor vehicle industry, household items and office items, and the sheets finding use in signs and displays, in the fields of transport, building, lights and sanitary ware, as noise walls, for works of art, flat screens, and the like.

Methyl methacrylate is also the starting material for the organic synthesis of higher methacrylates which, like it, are used in the preparation of acrylic emulsions and acrylic resins, act as additives for poly(vinyl chloride), are used as comonomers in the manufacture of numerous copolymers, such as methyl methacrylate/butadiene/styrene copolymers, act as additives for lubricants and have many other applications, among which may be mentioned medical prostheses, flocculants, cleaning products and the like. Acrylic emulsions and resins have applications in the field of paints, adhesives, paper, textiles, inks, and the like. Acrylic resins are also used in the manufacture of sheets having the same applications as PMMA.

Methyl methacrylate can be obtained in various ways, one of these consisting of an addition, at the alpha position, of methyl propionate to formaldehyde, according to the reaction:

CH₃—CH₂—COOCH₃+HCHO→CH₂═CH (CH₃)—COOCH₃

Methyl methacrylate can also be obtained by reacting methyl propionate with a formaldehyde/methanol mixture or also with methylal(dimethoxymethane CH₃OCH₂OCH₃), it being possible for the latter reaction to be catalyzed by a V/Si/P ternary oxide catalyst.

Reference may be made to pages 364-365 of the Encyclopedia of Chemical Technology, Kirk-Othmer, 3rd edition, vol. 15, which describe these synthetic routes.

Methyl propionate can be obtained by carbonylation of ethylene in the presence of methanol, by esterification of propionic acid by methanol or by hydrogenation of methyl acrylate. Propionic acid can be obtained by carbonylation of ethanol or by hydrogenation of acrylic acid. Methyl acrylate can be obtained by esterification of acrylic acid by methanol. Acrylic acid can be obtained by oxidation of acrolein, it being possible for the latter to be obtained by catalytic oxidation of propylene or by dehydration of glycerol, with production of acrylic acid as by-product.

The starting materials used for these syntheses of methyl methacrylate are mainly of petroleum origin or of synthetic origin, thus comprising numerous sources of emission of CO₂, which consequently contribute to increasing the greenhouse effect. For example, in the paper Industrial & Engineering Chemistry Research, 1997, 36(11), pp. 4600-4608, methyl methacrylate is manufactured by reaction of methyl propionate with formaldehyde, the methyl propionate having been obtained by a methoxycarbonylation reaction of ethylene with carbon monoxide originating from a syngas derived from coal (coal of fossil origin). Given the decrease in world oil reserves, the source of these starting materials will gradually become exhausted.

The starting materials resulting from biomass are a renewable source and have a reduced impact on the environment. They do not require all the stages of refining, which are very expensive in terms of energy, of oil products. The production of fossil CO₂ is reduced, so that they contribute less to climate warming. The plant, in particular for the growth thereof, has consumed atmospheric CO₂ at the rate of 44 g of CO₂ per mole of carbon (or per 12 g of carbon). Thus, the use of a renewable source begins by reducing the amount of atmospheric CO₂. Plant materials exhibit the advantage of being able to be cultivated in large amounts, according to demand, over most of the world.

It thus appears necessary to have available processes for the synthesis of methyl methacrylate which are not dependent on starting materials of fossil origin but which instead use biomass as starting material.

The term “biomass” is understood to mean starting material, of plant or animal origin, produced naturally. This plant material is characterized in that the plant, for its growth, has consumed atmospheric CO₂ for producing oxygen. Animals, for their growth, have for their part consumed this plant starting material and have thus taken in the carbon derived from atmospheric CO₂.

The aim of the present invention is thus to respond to certain concerns for sustainable development.

A subject matter of the present invention is thus a process for the manufacture of methyl methacrylate by reaction of methyl propionate with formaldehyde or a formaldehyde/methanol mixture or methylal, characterized in that at least a fraction of at least one reactant involved in this reaction was obtained by a reaction or a sequence of reactions starting from biomass.

It is thus possible to have obtained at least a fraction of the formaldehyde or at least a fraction of the methylal by oxidation of methanol, at least a fraction of the methanol involved having been obtained by pyrolysis of wood or by gasification of any material of animal or plant origin resulting in a syngas composed essentially of carbon monoxide and hydrogen, or by fermentation starting from plant crops, such as wheat, corn, sugar cane or beet, giving fermentable products and thus alcohol.

The reaction of methyl propionate with formaldehyde consists of a catalytic condensation in the gas phase, with a large excess of methyl propionate, optionally in the presence of methanol, at a temperature generally of between 225° C. and 450° C. Mention may be made, among effective catalysts, of alkali metal or alkaline earth metal aluminosilicates, or silica or alumina impregnated with a hydroxide, with a carbonate or with a nitrate, for example of potassium, of cesium or of zirconium, or of a lanthanide. Operating conditions for carrying out the reaction are described in particular in the documents FR 2 223 080 and U.S. Pat. No. 3,701,798.

The reaction of methyl propionate with methylal is carried out with an excess of methyl propionate, optionally in the presence of water, at a temperature generally of between 200° C. and 500° C., in the presence of a catalyst which can be chosen from magnesium, calcium, aluminum, zirconium, thorium and/or titanium phosphates and/or silicates, alone or with the addition of zirconium, aluminum, thorium and/or titanium oxides and/or of boric acid and/or of urea, it being possible for the catalyst to be modified by an alkali metal or alkaline earth metal carboxylate. Other catalytic systems can be used, for example, silica comprising a basic compound, which silica is combined with a catalyst comprising titanium dioxide. Operating conditions for carrying out the reaction are described in particular in the following documents FR 2 400 499, FR 2 347 330, FR 2 377 995 or GB 1491 183.

In accordance with a first embodiment, it is possible to have obtained at least a fraction of the methyl propionate by carbonylation of ethylene in the presence of methanol, at least a fraction of at least one from the ethylene, the carbon monoxide and the methanol involved in this methoxycarbonylation reaction having been obtained by a reaction or a sequence of reactions starting from biomass.

In particular, it is possible to have obtained at least a fraction of the ethylene by synthesis of ethanol by ethanolic fermentation of at least one plant material and optionally purification of the ethanol obtained, and then by dehydration of the ethanol obtained in order to produce a mixture of ethylene and water, removal of the water and optional purification of the ethylene obtained; and/or to have obtained at least a fraction of the carbon monoxide by gasification of any material of animal or plant origin, resulting in a syngas composed essentially of carbon monoxide and hydrogen, from which the carbon monoxide has been extracted; and/or to have obtained at least a fraction of the methanol by pyrolysis of wood or by gasification of any material of animal or plant origin, resulting in a syngas composed essentially of carbon monoxide and hydrogen, or by fermentation starting from plant crops, such as wheat, corn, sugar cane or beet, giving fermentable products and thus alcohol.

The plant material subjected to the ethanolic fermentation could advantageously have been chosen from sugars, starch and plant extracts comprising them, among which may be mentioned beet, sugar cane, cereals such as corn, wheat, barley and sorghum, potatoes, and a source of cellulose (mixture of cellulose, hemicellulose and lignin), but also organic waste. Ethanol is then obtained by fermentation, for example, using Saccharomyces cerevisiae or its mutant.

The dehydration of the ethanol could have been carried out using a catalyst based on γ-alumina.

In accordance with a second embodiment of the invention, it is possible to have obtained at least a fraction of the methyl propionate by esterification of propionic acid by methanol, at least a fraction of at least one from the propionic acid and the methanol involved in this reaction having been obtained by a reaction or a sequence of reactions starting from biomass.

In particular, it is possible to have obtained at least a fraction of the propionic acid by carbonylation of ethanol, at least a fraction of the carbon monoxide having been obtained by gasification of any material of animal or plant origin, resulting in a syngas composed essentially of carbon monoxide and hydrogen, from which the carbon monoxide was extracted; and at least a fraction of the ethanol having been obtained by fermentation of at least one plant material and optionally purification of the ethanol obtained; and/or it is possible to have obtained at least a fraction of the methanol by pyrolysis of wood or by gasification of any material of animal or plant origin resulting in a syngas composed essentially of carbon monoxide and hydrogen, or by fermentation starting from plant crops, such as wheat, corn, sugar cane or beet, giving fermentable products and thus alcohol.

In particular, it is also possible to have obtained at least a fraction of the propionic acid by hydrogenation of acrylic acid, the latter having been obtained as a by-product from the dehydration of glycerol. It was possible to obtain at least a fraction of the glycerol as by-product from the manufacture of biofuels starting from oleaginous plants, such as rape, sunflower or soya, comprising triglycerides, a hydrolysis or a transesterification of these triglycerides making it possible to form glycerol in addition to fatty acids and fatty esters respectively.

In accordance with a third embodiment of the invention, it was possible to obtain at least a fraction of the methyl propionate by hydrogenation of methyl acrylate, itself obtained by esterification of acrylic acid by methanol,

at least a fraction of the methanol having been obtained by pyrolysis of wood or by gasification of any material of animal or plant origin resulting in a syngas composed essentially of carbon monoxide and hydrogen, or by fermentation starting from plant crops, such as wheat, corn, sugar cane or beet, giving fermentable products and thus alcohol; and/or
at least a fraction of the acrylic acid having been obtained as by-product from the dehydration of glycerol, itself obtained as by-product from the manufacture of biofuels starting from oleaginous plants, such as rape, sunflower or soya.

Furthermore, it was possible to obtain at least a fraction of the methanol which has to react with the methyl propionate by pyrolysis of wood or by gasification of any material of animal or plant origin resulting in a syngas composed essentially of carbon monoxide and hydrogen, or by fermentation starting from plant crops, such as wheat, sugar cane or beet, giving fermentable products and thus alcohol.

In the various cases mentioned above, the syngas for preparing the methanol advantageously originates from the spent liquor from the manufacture and bleaching of cellulose pulps.

Another subject matter of the present invention is the use of the methyl methacrylate manufactured by the process as defined above as monomer for the manufacture of poly(methyl methacrylate), as starting material for the organic synthesis of higher methacrylates, as product used in the preparation of acrylic emulsions and acrylic resins, as additive for poly(vinyl chloride), as comonomer in the manufacture of copolymers, and as additive for lubricants.

### Recovery in Value of Biomass as Methanol

As indicated above, the methanol is obtained by pyrolysis of wood, by gasification of any material of animal or plant origin, resulting in a syngas composed essentially of carbon monoxide and hydrogen, which is optionally reacted with water in order to adjust the H₂/CO ratio to within the proportions appropriate to the synthesis of methanol, or by fermentation starting from plant crops, such as wheat, corn, sugar cane or beet, giving fermentable products and thus alcohol.

The materials of animal origin are, as non-limiting examples, fish oils and fats, such as cod liver oil, whale oil, sperm whale oil, dolphin oil, seal oil, sardine oil, herring oil or shark oil, oils and fats of bovines, porcines, caprines, equids, and poultry, such as tallow, lard, milk fat, pig fat, chicken, cow, pig or horse fats, and others.

The materials of plant origin are, as non-limiting examples, lignocellulose residues from agriculture, cereal straw fodder, such as wheat straw fodder or corn straw or ear residue fodder; cereal residues, such as corn residues; cereal flours, such as wheat flour; cereals, such as wheat, barley, sorghum or corn; wood, or wood waste and scraps; grains; sugar cane or sugar cane residues; pea tendrils and stems; beets or molasses, such as beet molasses; Jerusalem artichokes; potatoes, potato haulms or potato residues; starch; mixtures of cellulose, hemicellulose and lignin; and black liquor from the paper-making industry, which is a material rich in carbon.

According to a specific embodiment of the invention, the syngas for preparing the methanol originates from the recovery of spent liquor from the manufacture of cellulose pulps. Reference may be made to the documents EP 666 831 and U.S. Pat. No. 7,294,225 of Chemrec, which describe, in particular, the gasification of spent liquors from the manufacture and bleaching of cellulose and the production of methanol, and to pages 92-105 of the work Procédés de pétrochimie—Caractéristiques techniques et économiques—Tome 1—Editions Technip—le gaz de synthèse et ses dérivés [Petrochemical processes—Technical and Economic Characteristics—Volume 1—Published by Technip—Syngas and its derivatives], which relates to the production of methanol from syngas.

### Recovery in Value of Biomass as Ethylene

The ethylene is obtained by dehydration of ethanol, which is obtained by ethanolic fermentation of at least one plant material in the presence of one or more yeasts or mutants of these yeasts (microorganisms naturally modified in response to a chemical or physical stress), the fermentation being followed by distillation in order to recover the ethanol in the form of a more concentrated aqueous solution, which solution is subsequently treated for the purpose of further increasing the molar concentration thereof.

The plant material can be chosen in particular from sugars, starch and the plant extracts comprising them, among which may be mentioned beet, sugar cane, cereals, such as wheat, barley, sorghum or corn, and potatoes without this list being limiting. It can alternatively be biomass (mixture of cellulose, hemicellulose and lignin).

The plant material employed is generally in the form hydrolyzed before the fermentation stage. This preliminary hydrolysis stage thus makes possible, for example, the saccharification of the starch, in order to convert it into glucose, or the conversion of sucrose into glucose.

The ethanol is generally obtained as a mixture, with heavier alcohols, known as fusel alcohols, the composition of which depends on the plant material used and on the fermentation process. They generally comprise approximately 50% of isoamyl (C5) alcohol and a few percent of C3 and C4 alcohols (isobutanol). It is thus preferable according to the invention, to purify the ethanol produced by fermentation, for example, by distillation and/or absorption on filters of the molecular sieve, carbon black or zeolite type.

The ethanol obtained by fermentation and advantageously purified as indicated above is subsequently dehydrated in a reactor to give a mixture of ethylene and water. It is preferable for the ethanol to be injected at the top of the reactor.

This dehydration stage is generally carried in the presence of a catalyst which can be a γ-alumina. An example of a catalyst suitable for the dehydration of ethanol is sold in particular by Eurosupport under the trade name ESM 110®. It is an undoped trilobe alumina not comprising much residual Na₂O (usually 0.04%). A person skilled in the art will know how to choose the optimum operating conditions for this dehydration stage. By way of example, it has been shown that a ratio of the flow rate by volume of liquid ethanol to the volume of catalyst of 1 h⁻¹ and a mean temperature of the catalytic bed of 400° C. result in virtually complete conversion of the ethanol with a selectivity for ethylene of the order of 98%.

The ethylene obtained can optionally be composed of a mixture with other alkenes, if the ethanol was not purified as indicated above; in other words, if the ethanol was subjected to the dehydration as a mixture with fusel alcohols. It is therefore advantageous in this case to provide a stage of purification of the ethylene obtained, for example, by absorption on filters of molecular sieve, carbon black or zeolite type.

### Recovery in Value of Biomass as Carbon Monoxide

The carbon monoxide is obtained by gasification of any material of animal or plant origin, resulting in a syngas composed essentially of carbon monoxide and hydrogen, from which the carbon monoxide is extracted.

### Recovery in Value of Biomass as Glycerol

The glycerol is obtained from oleaginous plants, such as rape, sunflower or soya, comprising oils (triglycerides) or from animal fats.

A stage of hydrolysis or transesterification of the triglycerides is carried out in order to form, with the glycerol, fatty acids and fatty esters respectively.

For example, this transesterification can be carried out by reacting the crude oil in a stirred reactor in the presence of an excess of alcohol (for example methanol), preferably with a basic catalyst (such as sodium methoxide or sodium hydroxide). In order to carry out the hydrolysis reaction, the crude oil is reacted in the presence of an excess of water, preferably with an acid catalyst. This transesterification or hydrolysis reaction is preferably carried out at a temperature of between 40 and 120° C. Preferably, the reactor is fed continuously in order to keep the water/acid or alcohol/ester molar ratio greater than or equal to 2/1. At the end of the reaction, the glycerol is separated by settling from the mixture obtained.

The present invention thus makes it possible to obtain a methyl methacrylate having at least a portion of its carbons of renewable origin.

A renewable starting material or bioresource material is an animal or plant natural resource, the stock of which can be reconstituted over a short period on the human scale. In particular, it is necessary for the stock to be able to be renewed as quickly as it is consumed.

Unlike the materials resulting from fossil materials, renewable starting materials comprise ¹⁴C in the same proportions as atmospheric CO₂. All the samples of carbon drawn from living organisms (animals or plants) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the inorganic form, that is to say in the form of carbon dioxide gas (CO₂), and in the organic form, that is to say in the form of carbon incorporated in organic molecules.

In a living organism, the ¹⁴C/¹²C ratio is kept constant by the metabolism as the carbon is continually exchanged with the environment. As the proportion of ¹⁴C is constant in the atmosphere, it is the same in the organism, as long as it is living, since it absorbs this ¹⁴C as it absorbs the ¹²C. The mean ¹⁴C/ ¹²C ratio is equal to 1.2×10⁻¹² for a bioresource material, whereas a fossil starting material has a zero ratio. Carbon-14 results from the bombardment of atmospheric nitrogen (14) and is spontaneously oxidized with the oxygen of the air to give CO₂. In our human history, the ¹⁴CO₂ content increased as a result of atmospheric nuclear explosions but then has not ceased to decrease after the holding of these tests.

¹²C is stable, that is to say that the number of ¹²C atoms in a given sample is constant over time. ¹⁴C is for its part radioactive (each gram of carbon of a living being contains enough ¹⁴C isotopes to give 13.6 disintegrations per minute) and the number of such atoms in a sample decreases over time (t) according to the law:

n=no exp (−at),

in which:
no is the ¹⁴C number at the start (on the death of the creature, animal or plant),
n is the number of ¹⁴C atoms remaining after time t,
a is the disintegration constant (or radioactive constant); it is related to the half life.

The half-life (or period) is the period of time, at the end of which any number of radioactive nuclei or unstable particles of a given entity is reduced by half by disintegration; the half-life T_{1/2} is related to the disintegration constant a by the formula aT_{1/2}=ln 2. The half life of ¹⁴C has a value of 5730 years. In 50 000 years, the ¹⁴C content is less than 0.2% of the starting content and thus becomes difficult to detect.

Petroleum products or natural gas or also coal thus do not comprise ¹⁴C.

In view of the half-life (T_{1/2}) of ¹⁴C, the ¹⁴C content is substantially constant from the extraction of the renewable starting materials up to the manufacture of the methyl methacrylate according to the invention and even up to the end of its use.

The methyl methacrylate obtained according to the invention comprises organic carbon resulting from renewable starting materials; it is for this reason characterized in that it comprises ¹⁴C.

In particular, at least 1% by weight of the carbons of said methyl methacrylate is of renewable origin. Preferably, at least 20% of the carbons of said methyl methacrylate are of renewable origin. More preferably still, at least 40% of the carbons of said methyl methacrylate are of renewable origin. More particularly, at least 60% and even more specifically still at least 80% of the carbons of said methyl methacrylate are of renewable origin.

The methyl methacrylate obtained according to the invention comprises at least 0.01×10⁻¹⁰% by weight, preferably at least 0.2×10⁻¹⁰%, of ¹⁴C with regard to the total weight of carbon. More preferably still, said methyl methacrylate comprises at least 0.4×10⁻¹% of ¹⁴C, more particularly at least 0.7×10⁻¹⁰% of ¹⁴C and more specifically still at least 0.9×10⁻¹⁰% of ¹⁴C. Advantageously, the methyl methacrylate obtained according to the process according to the invention comprises from 0.2×10⁻¹⁰% to 1.2×10⁻¹⁰% by weight of ¹⁴C, with regard to the total weight of carbon.

In a preferred embodiment of the invention, the methyl methacrylate obtained according to the invention comprises 100% of organic carbon resulting from renewable starting materials and consequently 1.2×10⁻¹⁰% by weight of ¹⁴C, with regard to the total weight of carbon.

The ¹⁴C content of the methyl methacrylate can be measured, for example, according to the following techniques:
by liquid scintillation spectrometry: this method consists in counting the “β” particles resulting from the disintegration of the ¹⁴C. The β radiation resulting from a sample of known weight (known number of carbon atoms) is measured for a certain time. This “radioactivity” is proportional to the number of ¹⁴C atoms, which can thus be determined. The ¹⁴C present in the sample emits β radiation which, on contact with the liquid scintillant (scintillator) gives rise to photons. These photons have different energies (of between 0 and 156 keV) and form what is referred to as a ¹⁴C spectrum. According to two alternative forms of this method, the analysis relates either to the CO₂ produced beforehand by combustion of the carbon-comprising sample in an appropriate absorbing solution or to the benzene after prior conversion of the carbon-comprising sample to benzene.
by mass spectrometry: the sample is reduced to graphite or to CO₂ gas and analyzed in a mass spectrometer. This technique uses an accelerator and a mass spectrometer in order to separate the ¹⁴C ions from the ¹²C ions and thus to determine the ratio of the two isotopes.

These methods for measuring the ¹⁴C content of the materials are clearly described in the standard ASTM D 6866 (in particular D6866-06) and in the standard ASTMD 7026 (in particular 7026-04). These methods compare the data measured on the analyzed sample with the data of a reference sample of 100% renewable origin, to give the relative percentage of carbon of renewable origin in the sample.

The measurement method preferably used in the case of methyl methacrylate is the mass spectrometry described in the standard ASTM D6866-06.

The methyl methacrylate obtained according to the process according to the invention constitutes a starting material mainly comprising methyl methacrylate, in the sense that the product resulting from the process can comprise impurities related to the nature of the reactants employed or generated during the process, which can be different from the impurities generated during the use of reactants of fossil origin. The process according to the invention can thus comprise, in addition, one or more purification stages.

The methyl methacrylate obtained according to the process according the invention can be used, as is or optionally after a purification stage, as starting material in all the applications in which the use of MMA is known, in particular as monomer for the manufacture of poly(methyl methacrylate), as starting material for the organic synthesis of higher methacrylates, as product used in the preparation of acrylic emulsions and acrylic resins, as additive for poly(vinyl chloride), as comonomer in the manufacture of copolymers and as additive for lubricants.

The following examples illustrate the present invention without, however, limiting the scope thereof. In these examples, the parts and percentages are by weight, unless otherwise indicated.

### EXAMPLE 1

### Manufacture of Methyl Methacrylate by Reaction of Methyl Propionate with a Formaldehyde/Methanol Mixture

### 1—Manufacture of Methyl Propionate

### 1a—Preparation of the Ethanol

By Ethanolic Fermentation of Sugar

A water/sugar (10 kg of sugar) mixture is poured into a liter plastic tank. 0.25 l of baker's yeast mixed beforehand with 0.25 l of tepid water, and a dose of Calgon (water softener) are added to the mixture and the combined product is allowed to soak at a temperature of 25° C. for 14 days. In order to limit the formation of acetic acid, the container is covered with a lid provided with a valve. On conclusion of this stage, the mixture is filtered and separated by settling, and the solution is distilled in order to recover the azeotrope of the ethanol, at 96% in water.

By Ethanolic Fermentation of Corn Grains

Use is made of corn grains, which are placed in a container and covered with hot water. A cloth is placed over the container in order to eliminate contamination and heat losses. The container is provided with an orifice at the bottom in order to make possible slow flow. Hot water is regularly added in order to maintain the level. The container is thus maintained for 3 days, or until the grains have sufficiently exploded.

Subsequently, the grains are dried and are then ground. A slurry is prepared by adding hot water and it is thus maintained in order to start the fermentation. A yeast is added for the fermentation (250 g of yeast per 200 liters of slurry, for example) and optionally sugar. With the yeast, the fermentation takes approximately 3 days; in the absence of yeast, it can take 10 days. Use is made of a Saccharomyces cerevisiae yeast. The slurry is converted when it stops bubbling. The fermentation produces both ethanol and CO₂. The product is placed in a distillation vessel equipped with a distillation column. The first fractions dissolved comprise volatile contaminants and alcohol, and are discarded. Subsequently, the ethanol is collected. The final fractions are poor in alcohol.

### 1b—Manufacture of Ethylene by Dehydration of the Ethanol

In a plant, 96% ethanol, obtained by ethanolic fermentation of corn grains or of sugar as described above, is evaporated in an evaporator and then preheated in a heat exchanger before being injected at the top of a reactor with a diameter of 127 mm containing a catalytic bed brought to 300-400° C. and consisting of a layer of ESM110® alumina from Eurosupport, representing a volume of 12 700 cm³ and a weight of 6500 g, the ratio of the flow rate by volume of ethanol to the volume of catalyst being 1 h⁻¹. The mixture of water and ethylene produced in the reactor is cooled in the heat exchanger before being sent to a gas/liquid separator, where the ethylene and the water (possibly mixed with by-products) are separated.

### b 1c—Manufacture of Methyl Propionate by Carbonylation of the Ethylene in the Presence of Methanol

A solid palladium-based catalyst: palladium[bis(di(t-butyl)phosphine)-o-xylene]dibenzylideneacetone (37 mg, 5.0×10⁻⁵ mol), cobalt carbonyl (9 mg, 2.6×10⁻⁵ mol) and methanesulfonic acid produced by Arkema (68 microliters, 1.0×10⁻³ mol) are dissolved in methanol (219 ml, 5.41 mol) and methyl propionate (81 ml, 0.841 mol) under a nitrogen atmosphere. The solution is transferred into an autoclave and heated to 80° C., and then CO and the ethylene prepared above, in a molar ratio of 1:1, are continuously introduced into the reactor at a total pressure of 10 bar.

The reaction is carried out for a time of 4 hours and the products are analyzed in order to determine the amount of methyl propionate formed: i.e. 4329 kg of methyl propionate per kg of palladium and per hour. The reaction yield with respect to the methanol involved is 19%. In this reactor configuration, the unconverted ethylene and the unconverted CO are recycled and the methanol remains in the reactor.

The methyl propionate is subsequently isolated for the following stage.

### 2—Manufacture of Methyl Methacrylate by Reaction of Methyl Propionate with Formaldehyde and Methanol

In this example, use is made of a catalyst of CS/Zr/SiO₂ type prepared from a silica gel in the form of spheres with a diameter of 2-4 mm having a purity of 99.9%, a specific surface of 320 m²/g and a pore volume of 0.83 cm³/g with a median pore diameter of 9 nm.

The silica is impregnated with an aqueous zirconium nitrate solution (impregnation with interaction), filtered off and dried in a rotary evaporator and then an oven at 120° C. for 2 hours. The impregnation and the drying were repeated a further two times, so as to obtain a deposition of 0.02% by weight (1.2 g of zirconium per 100 mol of silica). The cesium is then itself also impregnated starting from an aqueous cesium carbonate solution, followed by drying to give a cesium content of approximately 4% by weight (calculated as weight of metal). The catalyst was then calcined at 450° C. under air for 3 hours. The specific surface of the catalyst thus prepared is 300 m²/g.

Use is made of methanol originating from the reaction of a syngas obtained by gasification of black liquor.

The reaction was carried out in a microreactor at atmospheric pressure, charged with approximately 3 g of ground catalyst, in order to have particles of the order of a millimeter. The catalyst is first of all dried at 300° C., for 30 minutes under a stream of 100 ml/min of nitrogen. The catalyst is heated to 300° C. and fed with a mixture of methyl propionate, methanol and formaldehyde solution (formaldehyde/methanol/water: 35/15/50—ratios by weight), so that the methanol/methyl propionate and formaldehyde/methyl propionate molar ratios are 1.45 and 0.187 respectively.

After a stabilization period of 30 minutes with a contact time of 5 s, the temperature of the catalyst is brought to 350° C. overnight. After this optional stage of conditioning, the methyl methacrylate+methacrylic acid yield is 9%, with a selectivity of 97%.

### EXAMPLE 2

### Manufacture of Syngas CO/H₂ and Isolation of the Carbon Monoxide

In the process of the synthesis of methyl propionate, it is not necessary to look for high carbon monoxide purities and in particular it is possible to have residual nitrogen as the pressures at which the process is carried out are relatively low. However, any inert impurity, such as nitrogen or argon, which cannot be consumed by the reaction, will gradually contribute to a diluting of the ethylene and CO. Although nitrogen and argon are not harmful chemically in the process, it is therefore preferable to limit as far as possible the content of these impurities.

The pressure at which the carbon monoxide is used subsequently is also relatively low; nevertheless, as the purification treatments result in pressure drops, it is preferable to carry out the gasification of biomass under pressure.

In the present example, use is made of an ethanol/water mixture, the ethanol being obtained by fermentation, as in example 1a. The operation is carried out under a pressure of 30 bar and at a temperature of 900° C., with an Ni/alumina catalyst. At the outlet of the reactor, the excess water is condensed, along with the heavy impurities.

The CO/H₂ mixture is separated cryogenically, the mixture being passed into a liquid nitrogen trap in order to retain the CO. The condensed gas is subsequently reheated in order to separate the CO from the other impurities (methane, CO₂, and the like).

### EXAMPLE 3

### Manufacture of Methanol from Syngas

For the synthesis of methanol, use is made of syngas from example 2. The composition of this gas is adjusted in order to have an H₂/CO/CO₂ ratio of 71/23/6 and the CO₂ content is 6%. The total pressure of gas is 70 bar.

Use is made of a commercial Cu/Zn/Al/O catalyst MegaMax 700. The reactor is fed with the gas mixture at 70 bar with an HSV of 10 000 h⁻¹, which mixture passes over the catalyst at a temperature of 240° C. The mixture of the gases produced is subsequently reduced in pressure to atmospheric pressure and the methanol produced is isolated by distillation.

The selectivity for methanol is 99% and the methanol yield is 95%.

### EXAMPLE 4

### Manufacture of Formaldehyde by Oxidation of Methanol

The reaction is carried out in a fixed bed reactor. The stream of helium and oxygen is regulated by mass flowmeters. The gas stream passes through an evaporator/saturator containing the methanol prepared according to example 3. The evaporator is either at ambient temperature or heated by heating tapes. The temperature of the saturator is adjusted in order to control the methanol partial pressure. The temperature of the gas mixture is controlled by a thermocouple at the top of the saturator.

The gas mixture is subsequently sent to the reactor, which is placed in an oven. The reaction temperature is measured using a thermocouple which is in the catalytic bed.

The gas outlet flows are analyzed by in-line gas chromatography using a MicroGC equipped with two columns (molecular sieve and Plot U).

The catalysts are ground and the fraction with a particle size of 250 microns is mixed with a two-fold amount of silicon carbide with the same particle size and placed in the glass reactors.

The calibration of the MicroGC is carried out with mixtures of the reference gases and the calibration for the condensable products (dimethoxymethane, methanol, methyl formate) is carried out using the evaporator/saturator.

151 mg of an iron molybdate catalyst MFM3-MS (external diameter=3.9 mm, internal diameter 1.85 mm, height=4.04 mm) supplied by MAPCO are mixed with 300 mg of silicon carbide and charged to the reactor.

The catalyst is first of all activated under a helium/oxygen stream (48 Sml/min−12 Sml/min) at 340° C. for 15 hours 30 minutes. Subsequently, the temperature is brought back to 280° C. and the accumulation of the product is begun.

The oxygen and helium flow rates are 4.7 and 47.6 Sml/min respectively and the concentration of the methanol is adjusted to 5% of the reaction medium (methanol/O₂/inert material: 5/8.5/86.5).

Virtually all the methanol is converted and the formaldehyde selectivity is 90%. The products are recovered at the outlet of the reactor in a thermostatically controlled cold trap. The product obtained is subsequently passed through an anionic resin, in order to remove the acids present, and an aqueous solution of methanol is added in order to obtain a standard formaldehyde composition with formaldehyde/water/methanol ratios by weight of 35/50/15. The methanol added inhibits the reactions of the formaldehyde and thus prevents the resulting formation of by-products, such as hemiacetals and polyacetals.

### EXAMPLE 5

### Manufacture of Methylal by Oxidation of Methanol

Example 4 is repeated but with the following conditions:

The catalyst is first of all activated under a helium/oxygen stream (48 Sml/min−12 Sml/min) at 340° C. for 15 hours 30 minutes. Subsequently, the temperature is brought back to 250° C. After stabilization, the products are accumulated. Subsequently, the temperature of the catalyst is increased stepwise up to 280° C.

The oxygen and helium flow rates are 6.7 and 26.4 Sml/min respectively and the concentration of the methanol is adjusted to 37% (conditions: methanol/O₂/inert material: 37/13/50) for an HSV of 22 000 ml·h⁻¹·g⁻¹.

The results for conversions and selectivities obtained during the catalytic oxidation of the methanol are as follows:

Conversion: 55.7%

Selectivities: methylal 89.8%
formaldehyde 4.2%
dimethyl ether 5.3%
methyl formate 0.6%

The methylal is subsequently separated by distillation of the other products, its azeotrope with water being obtained.

### EXAMPLE 6

### Synthesis of a 100% Renewable Methyl Methacrylate by Reaction of Methyl Propionate with Formaldehyde and Methanol

The methanol of example 3 and the CO of example 2 are used in conjunction with the ethylene of example 1-1b to produce, under the conditions of example 1-1c, 100% renewable methyl propionate.

In the first stage, a methyl propionate yield of 18%, with respect to the methanol is obtained.

After obtaining methyl propionate, the latter is reacted with formaldehyde (obtained as in example 4) and methanol (obtained as in example 3) under the conditions of example 1-2.

A methyl methacrylate+methacrylic acid yield of 8% is obtained.

### EXAMPLE 7

### Synthesis of Methyl Methacrylate by Reaction of Propionaldehyde and Methylal

Example 6 is repeated but methylal (obtained as in example 5) is used in place of the methanol/formaldehyde mixture, while retaining the molar ratios.

The methyl methacrylate+methacrylic acid yield is 6% and the selectivity is 94%.

1. A process for the manufacture of methyl methacrylate comprising reacting methyl propionate with one from formaldehyde, a formaldehyde/methanol mixture and methylal, wherein at least a fraction of at least one reactant involved in the reaction of methyl propionate with one of formaldehyde, a formaldehyde/methanol mixture and methylal was obtained by a reaction or a sequence of reactions starting from biomass.

2. The process as claimed in claim 1, wherein at least a fraction of the formaldehyde or at least a fraction of the methylal was obtained by oxidation of methanol, at least a fraction of the methanol involved having been obtained by pyrolysis of wood or by gasification of any material of animal or plant origin resulting in a syngas consisting essentially of carbon monoxide and hydrogen, or by fermentation starting from plant crops giving fermentable products and alcohol.

3. The process as claimed in claim 1, characterized wherein at least a fraction of the methyl propionate was obtained by carbonylation of ethylene in the presence of methanol, at least a fraction of at least one from the ethylene, the carbon monoxide and the methanol involved in the methoxycarbonylation reaction having been obtained by a reaction or a sequence of reactions starting from biomass.

4. The process as claimed in claim 3, wherein at least a fraction of the ethylene was obtained by synthesis of ethanol by ethanolic fermentation of at least one plant material and optionally purification of the ethanol obtained, and then by dehydration of the ethanol obtained in order to produce a mixture of ethylene and water, removal of the water and optional purification of the ethylene obtained; and/or in that at least a fraction of the carbon monoxide was obtained by gasification of any material of animal or plant origin, resulting in a syngas consisting essentially of carbon monoxide and hydrogen, from which the carbon monoxide has been extracted; and/or
in that at least a fraction of the methanol was obtained by pyrolysis of wood or by gasification of any material of animal or plant origin, resulting in a syngas consisting essentially of carbon monoxide and hydrogen, or by fermentation starting from plant crops, giving fermentable products and alcohol.

5. The process as claimed in claim 4, wherein the plant material subjected to the ethanolic fermentation was chosen from sugars, starch and plant extracts comprising them.

6. The process as claimed in claim 4, wherein the dehydration of the ethanol was carried out using a catalyst based on γ-alumina.

7. The process as claimed in claim 1, wherein at least a fraction of the methyl propionate was obtained by esterification of propionic acid by methanol, at least a fraction of at least one from the propionic acid and the methanol involved in this reaction having been obtained by a reaction or a sequence of reactions starting from biomass.

8. The process as claimed in claim 7, wherein at least a fraction of the propionic acid was obtained by carbonylation of ethanol, at least a fraction of the carbon monoxide having been obtained by gasification of any material of animal or plant origin, resulting in a syngas consisting essentially of carbon monoxide and hydrogen, from which the carbon monoxide was extracted; and/or
at least a fraction of the ethanol having been obtained by fermentation of at least one plant material and optionally purification of the ethanol obtained; and/or
in that at least a fraction of the methanol was obtained by pyrolysis of wood or by gasification of any material of animal or plant origin resulting in a syngas consisting essentially of carbon monoxide and hydrogen, or by fermentation starting from plant crops, giving fermentable products and thus alcohol.

9. The process as claimed in claim 7, wherein at least a fraction of the propionic acid was obtained by hydrogenation of acrylic acid, the acrylic acid having been obtained as a by-product from the dehydration of glycerol.

10. The process as claimed in claim 9, wherein at least a fraction of the glycerol was obtained as by-product from the manufacture of biofuels starting from oleaginous plants comprising triglycerides, wherein a hydrolysis or a transesterification of the triglycerides form glycerol, in addition to fatty acids and fatty esters.

11. The process as claimed in claim 1, wherein at least a fraction of the methyl propionate was obtained by hydrogenation of methyl acrylate, itself obtained by esterification of acrylic acid by methanol,
at least a fraction of the methanol having been obtained by pyrolysis of wood or by gasification of any material of animal or plant origin resulting in a syngas consisting essentially of carbon monoxide and hydrogen, or by fermentation starting from plant crops, giving fermentable products and thus alcohol; and/or
at least a fraction of the acrylic acid having been obtained as by-product from the dehydration of glycerol, itself obtained as by-product from the manufacture of biofuels starting from plants.

12. The process as claimed in claim 1, wherein at least a fraction of the methanol which has to react with the methyl propionate was obtained by pyrolysis of wood or by gasification of any material of arimal or plant origin, resulting in a syngas consisting essentially of carbon monoxide and hydrogen, or by fermentation starting from plant crops, giving fermentable products and alcohol.

13. The process as claimed in claim 4 wherein the syngas for preparing the methanol originates from the spent liquor from the manufacture and bleaching of cellulose pulps.

14. A method of using methyl methacrylate comprising from 0.2×10⁻¹⁰% to 1.2×10⁻¹⁰% by weight of ¹⁴C with regard to the total weight of carbon manufactured by the process as defined in claim 1, comprising at least one of:
using the methyl methacrylate as a monomer for the manufacture of poly(methyl methacrylate),
using the methyl methacrylate as a starting material for the organic synthesis of higher methacrylates,
using the methyl methacrylate as a product used in the preparation of acrylic emulsions and acrylic resins,
using the methyl methacrylate as an additive for poly(vinyl chloride),
using the methyl methacrylate as a comonomer in the manufacture of copolymers, and
using the methyl methacrylate as an additive for lubricants.

15. The process as claimed in claim 5, wherein the plant material subjected to the ethanolic fermentation was chosen from beet, sugar cane, cereals, potatoes, a source of cellulose, or organic waste.

16. A method of manufacturing methyl methacrylate comprising:
(a) obtaining at least a fraction of at least one reactant selected from the group consisting of methyl propionate, formaldehyde, a formaldehyde/methanol mixture, and methylal by a reaction or a sequence of reactions starting from a biomass source; and
(b) subsequently, reacting methyl propionate with at least one of formaldehyde, a formaldehyde/methanol mixture, and methylal to form methyl methacrylate,
wherein at least a fraction of the methyl propionate, the formaldehyde, the formaldehyde/methanol mixture, or the methylal is the reactant obtained in step (a).

17. A method of manufacturing methyl methacrylate according to claim 16, wherein the methyl methacrylate comprises ¹⁴C.

18. A method of manufacturing methyl methacrylate according to claim 16, wherein the biomass source is a bioresource material having a mean ¹⁴C/¹²C ratio of about 1.2×10⁻¹².
